(19) United States
(12) Patent Application Publication
(10) Pub. No.: US 2012/0244227 A1
(43) Pub. Date: Sep. 27, 2012

(54) BIOLOGICALLY ACTIVE FOOD ADDITIVE FOR THE PROPHYLAXIS OF OSTEOPOROSIS DISEASE

(76) Inventor: Dmitriy Gennadievich Elistratov, Penza (RU)

(21) Appl. No.: 13/512,671

(22) PCT Filed: Oct. 21, 2010
(86) PCT No.: PCT/RU10/00613

§ 371 Date: May 30, 2012

(30) Foreign Application Priority Data
Nov. 30, 2009 (RU) 2009144461

(51) Int. Cl. A61K 35/64; A61P 19/10

(52) U.S. Cl. 424/539

(57) ABSTRACT

The invention relates to biologically active food supplements and is intended for preventive action against the states associated with osteoporosis. The technical effect consists in providing a biologically active food supplement which sustains a normal testosterone level in the body, ensures an efficient assimilation of calcium by the body and retention of calcium in the bony tissue for a prolonged time enabling thereby the prevention of osteoporosis.

### CROSS-REFERENCE TO RELATED APPLICATIONS

This application is a U.S. National phase application of the International application PCT/RU2010/000613, filed Oct. 21, 2010 claiming priority to Russian application 2009144461, filed Nov. 30, 2009, the entire contents of each of the applications being hereby incorporated into the present application by reference in full.

### BACKGROUND OF THE INVENTION

1. Field of the Invention

The present invention relates to biologically active food supplements and is intended for preventive action against the states associated with osteoporosis.

2. Description of Related Art

The problem of osteoporosis (reduction in the amount of calcium in the bone structures) is a common knowledge. In order to address this problem, calcium preparations are used based on calcium carbonate, calcium citrate, calcium gluconate, calcium aspartate, calcium ascorbate, calcium aminochelate, calcium fumarate, calcium succinate, calcium phosphate, calcium citrate and other compounds. It is known that calcium preparations are better assimilated in combination with vitamin D. Therefore, multiple drugs are known, for example, calcium gluconate tablets (registration number R No. 000140/02-2001 MZ RF), Calcium-D3 Nycomed tablets (registration number P No. 013478/01-2001), and the like.

The prior art preparations have the following drawbacks.

1) Unfortunately, all known preparations are directed only to increasing the ingress of calcium into the body rather than eliminating the reasons of the bones losing calcium.
2) When a calcium preparation is administered, calcium may enter not only the bones but also other organs and systems, in particular it may calcify concrements in the kidneys that leads to a malfunction of kidneys.

In other words, to treat osteoporosis with calcium preparations is as effective as trying to pour water into holed barrel. The ability of the body to assimilate calcium and to use the same as intended is believed to be more important than the amount of calcium entering the body.

On the other hand, it is known that an important role in preserving the density of the mineral bone stock in both young and elderly men is played by androgens, i.e. male sex hormones. A lower bone density and a higher incidence of bone fractures is observed in men suffering from hypogonadism (i.e. a reduced level of testosterone, a male sex hormone). A low testosterone level is considered one of the major causes of osteoporosis, i.e. lowered mineral bone density in men, and is hence a risk factor of fractures.

An average testosterone level in women is 25 times lower than that in men so that even an insignificant decrease in the testosterone level leads to osteoporosis. This also explains why more women than men suffer from osteoporosis.

Male sex hormones or androgens (first of all, testosterone) influence the bony tissue both directly and indirectly. The direct influence of testosterone on the bony tissue is responsible for the differences in the structure of male and female skeletons. Testosterone indirectly affects the bone formation through estrogens, female sex hormones which are formed from androgens under the action of enzyme aromatase. Since testosterone is the primary estrogen in males, a decrease in its secretion also leads to a decrease in the bone density.

Accordingly, men with diagnosed osteoporosis are supposed to undergo hormonal examination including tests for testosterone, estradiol and sex steroid-binding globulin. In turn, all men with a low content of sex hormones need to undergo densitometry with a view to detecting osteoporosis.

Various medicaments, such as calcium preparations, vitamins D H bisphosphonates, are used for the treatment of decreased bone density and osteoporosis. However, it has been proved that such medicaments have a minimum efficiency given that the testosterone level is still low, because the proper assimilation of medicaments directed to repairing the bone density is ensured precisely by testosterone. Therefore, the treatment of the bone density disorders has to be comprehensive, directed to both compensating for a deficit (if any) in testosterone and administering of calcium preparations.

However, when reducing this theory to practice problems have been faced with the known preparations sustaining the testosterone level in the body (for example, testosterone propionate) since they all require the most stringent health control.

The reason is that testosterone preparations are referred to doping drugs and have a lot of negative effects on the human body. When testosterone comes from the outside, the production of its own testosterone in the human body further decreases. On the other hand, as shown above, the body is in constant need for the ingress of calcium.

### SUMMARY OF THE INVENTION

Therefore, it is an object of the present invention to provide a biologically active food supplement for ongoing administration, which would compensate for the content of calcium in the body, sustain the testosterone level and also facilitate a better assimilation of calcium and retention thereof in the body.

This object is achieved by providing a biologically active food supplement for the prevention of osteoporosis, comprising on the weight percent basis:

a calcium compound selected from calcium carbonate, calcium citrate, calcium gluconate, calcium aspartate, calcium phosphate and calcium citrate—16.67 to 93.75 wt. %;

a drone brood—6.25 to 83.33 wt. %.

The claimed biologically active food supplement is available in a powdered, tablet or capsular form.

The technical effect consists in providing a biologically active food supplement which sustains a normal testosterone level in the body, to ensure the efficient assimilation of calcium by the body and retention thereof in the bony tissue for a prolonged time enabling thereby the prevention of osteoporosis.

### DETAILED DESCRIPTION OF THE INVENTION

The use of drone brood in the present invention can be explained by the following. The drone brood is a donator of sex entomological hormones such as prolactin, estradiol, progesterone, testosterone which stimulate the reproductive functions in men and women. Rich in hormones and vitamins not being hormone substitutes, the drone brood is effective against the hormonal background disorders, stimulates the central mechanisms for regulating the rate of androgen formation and excludes possible replacement therapy. The studies conducted in the Apiculture Scientific-Research Institute (Rybnoye, Ryazan region, Russia) demonstrated the safe use of the drone brood and also proved the gonadotropic effect of the drone brood on the stimulation of central links controlling the testosterone synthesis. (Krivtsov N. I. et al., Theory and Methods of Apitherapy, Moscow, 2007). Therefore, the testosterone level in the body may be sustained using the drone brood.

Although the components forming part of the inventive biologically active food supplement are known in the folk or traditional medicine, their combination in one product is not disclosed in the prior art.

The combined use of the mentioned components enables a more efficient assimilation of calcium by the body since calcium not only enters the body but is also retained in the bony tissue thanks to the drone brood. It has been found that the drone brood is able to improve the assimilability of calcium by the body and to retain the same in the bony tissue for a long time, i.e. to ensure a prolonged effect of the bony tissue calcium intake. In addition, the administration of calcium together with the drone brood makes it possible to lower the dosage of the calcium compounds, to thus prevent lithogenesis in the kidneys as well as other negative effects of taking the calcium preparations (for example, gastroenteric upsets) from occurring.

The ratio of the calcium compound and the drone brood varies within a wide range. The range width is determined by the individual parameters: age, gender, state of health and other factors.

It is known that the upper acceptance limit for the administration of calcium is 1000-1500 mg per day. Due to the combined administration of the drone brood and the calcium compound, the daily dosage of calcium may be lowered since the bioavailability (assimilability) of calcium is improved and bone resorption (calcium depletion) is prevented.

Based on the foregoing, the maximum consumption of calcium is suggested to be 600 mg.

The minimum consumption of calcium is determined by the minimum body need for ionized calcium, that being of 200 mg.

The maximum consumption of the drone brood is determined by the expediency of use and is 1000 mg.

The minimum consumption of the drone brood is 40 mg (see RU2233666 C1, Oct. 8, 2004) and is determined by the pharmacological activity.

Therefore, the administration range of the components forming the biologically active food supplement according to the present invention is as follows:

drone brood: 40 to 1000 mg or 6.25% to 83.33%;

calcium: 200 to 600 mg or 16.67% to 93.75%.

Therefore, the combined use of the drone brood and calcium compound in the supplement makes it possible to reduce significantly the maximum (upper) daily consumption of calcium from 1500 mg to 600 mg thereby reducing the negative side effects of calcium on the human body and widening the group of persons able to use calcium preparations. The reduction in the maximum daily consumption of calcium is necessary because an opposite disease, hypercalcemia, quiet often occurs in cases where calcium preparations are administered in the doses of 1000 to 1500 mg. For example, of the 1000 patients continuously receiving preparations against osteoporosis, 86 had hypermineralization of the 1 to 3 degree (Strukov V. I., Actual Problems of Osteoporosis, Penza, 2009, Rostra Publishers).

The above-identified quantitative ratio makes it possible to ensure an optimum ingress of calcium for each individual and its most complete assimilation thanks to the claimed amount of the drone brood which ensures a certain content of sulfhydryl enzyme groups in the supplement, as well as testosterone, progesterone, prolactin and estradiol. The drone brood is rich in amino acids. The amino acids in turn act as transporters of calcium to the cell (Strukov V. I., Ibid.). In order for calcium to be assimilated, vitamins A, C, E, D and micronutrient elements, such as magnesium, copper, zinc, phosphorus are also required. All of them are present in the drone brood.

As a result, all calcium being administered is completely assimilated by the bony tissue. Surprisingly, it has been found that the retention of calcium in the bony tissue for a prolonged time is provided by a certain amount of sulfhydryl enzyme groups in the drone brood, vitamins D and E so that the process of its depletion slows down. Besides, a long period of the normal bony tissue density is accounted for by a sufficiently high content of phosphorus in the drone brood which enters the body in the amount specified above, thereby facilitating the assimilation of calcium.

Therefore, the problem of not only saturating the bony tissue with calcium but also of retaining the same for a long time therein has been solved for the first time by means of the combined use of the calcium preparations and the drone brood in the claimed ratio. The risk of fractures has been considerably lessened as compared to the prior art preparations since a 5% increase in the mineral bone density leads to a 35% reduction in the risk of fracture, and the additional antiresorptive feature of the claimed preparation reduces the risk of fracture by another 20%.

In order to prepare the biologically active food supplement in accordance with the present invention, there is provided a powdered calcium compound selected from calcium carbonate, calcium citrate, calcium gluconate, calcium aspartate, calcium phosphate, calcium citrate at a concentration of 16.67 wt. % to 93.75 wt. % and is mixed with a drone brood at a concentration of 6.25 wt. % to 83.33 wt. % until a homogeneous mixture is formed. The resulting mixture is then tableted or capsulated. The biologically active food supplement presents a homogeneous light beige powder with a humidity of 1 to 3.5%, which is further subjected to tableting or capsulation. A tablet or a capsule contains 1 g of the mixture.

When preparing the biologically active food supplement, the drone brood is used in the form of a lyophilizate. In order to obtain a lyophilizate, the larval drone brood is homogenized using a mixer. The homogenate comprises a thick yellow creamy liquid with a characteristic odor. The content of dry matter in the homogenate does not exceed 22.5%. The lyophilizate is obtained by freezing the homogenate for 2-3 hours at a temperature of minus 35-40° C. followed by vacuum sublimation at a residual pressure of 0.1-0.6 mm hg for 40-48 hours and bringing the temperature to 25-30° C. by the end of lyophilization. The lyophilizate includes a powder whose color varies from beige to yellow, with a high hygroscopicity and residual moisture content of 1-3.5%. In other words, the preparation conditions of lyophilizate are such that the solid matter composition of the drone brood homogenate may be preserved without changes except for the moisture content.

Below are provided the examples of preparing the biologically active food supplement.

### Example 1

17 kg of powdered calcium gluconate was provided and mixed with 83 kg of the drone brood lyophilizate. The mixture was then stirred to homogeneity and the resulting product was capsulated.

### Example 2

90 kg of powdered calcium carbonate was provided and mixed with 10 kg of the drone brood lyophilizate. The mixture was stirred to homogeneity and then tableted.

For prevention of osteoporosis, one capsule of the biologically active food supplement is administered 3 times a day for one month separately from meals. A one-month break in the administration should be provided. The serum calcium value does not reduce during this period. In other words, the calcium content is sustained at a normal level.

The above technical effect is demonstrated by way of the following examples.

### Example 1

A human subject, female, born in 1959. The mineral bone density of T −1.4 was measured in the distal antibrachium by means of the forearm scanner DXT-200 available from Osteometer MediTech, Inc. Osteopenia was diagnosed and 1 g of calcium gluconate was administered 3 times a day. A six-month course of preventive medication was conducted. A re-evaluation six months later showed a reduced bone density down to T −1.6. That is, the mineral bone density (MBD) became less. After a one-month break in the administration, the serum calcium value was below the normal value.

For reference: MBD normal value—the values of T criterion with a standard deviation of +1 to −1 from the peak bone mass;

1^{st} degree osteopenia—MBD of −1 to −1.5;
2^{nd} Z degree osteopenia—MBD of −1.5 to −2;
3^{nd} degree osteopenia—MBD of −2 to −2.5;
1^{st} degree osteoporosis—MBD of −2.5 and less without fractures;
2^{nd} degree osteoporosis—MBD of −2.5 and less with osteoporotic bone fractures.

The densitometric T scale presents the number of standard deviations up and down an average bone mass value. The T criterion decreases in parallel with a gradual reduction in the bone mass with age and is used to evaluate the MBD in adults.

### Example 2

The same female human subject with diagnosed osteopenia and the mineral bone density of T −1.6 was administered one capsule of the claimed biologically active food supplement 3 times a day. A six-month preventive medication course was conducted following the pattern of one-month administration/one-month break. The mineral bone density increased to T −1.2. The serum calcium value normalized. No reduction in the serum calcium value was observed during the re-examination performed three months later.

Therefore, apart from sustaining the normal testosterone level and compensating for the deficit of calcium in the body, the inventive biologically active food supplement according to the present invention also facilitates a more efficient assimilation of calcium and retention thereof in the bony tissue for a long time (i.e. has a prolonged activity) and thus is believed to be an excellent preparation for the prevention of osteoporosis.

1. (canceled)

2. A biologically active food supplement intended for the prevention of osteoporosis, comprising on the weight percent basis:
a calcium compound—16.67 to 93.75;
drone brood—6.25 to 83.33.

3. The biologically active food supplement as claimed in claim 2, wherein the calcium compound is selected from the range comprising calcium carbonate, calcium citrate, calcium gluconate, calcium aspartate, calcium phosphate, calcium citrate.

4. The biologically active food supplement as claimed in claim 2, wherein the supplement is made in a powdered form.

5. The biologically active food supplement as claimed in claim 2, wherein the supplement is made in a tableted form.

6. The biologically active food supplement as claimed in claim 2, wherein the supplement is made in a capsulated form.

7. A biologically active food supplement intended for the prevention of osteoporosis, comprising on the weight percent basis:
a calcium compound selected from the range comprising calcium carbonate, calcium citrate, calcium gluconate, calcium aspartate, calcium phosphate, calcium citrate—16.67 to 93.75;
drone brood—6.25 to 83.33, the supplement being made in a powdered, tableted or capsulated form.

8. A method of preparing a biologically active food supplement for the prevention of osteoporosis comprising the steps of:
providing a calcium compound selected from a range comprising calcium carbonate, calcium citrate, calcium gluconate, calcium aspartate, calcium phosphate, calcium citrate;
providing drone brood in the form of lyophilizate,
the calcium compound and drone brood being taken at weight percent concentration of 16.67-93.75 for calcium compound and 6.25-83.33 for the drone brood;
mixing the calcium compound and drone brood until a homogeneous mixture is obtained in a powder form adapted to be tableted or capsulated.

9. The method of preparing a biologically active food supplement for the prevention of osteoporosis as claimed in claim 8, wherein the drone brood lyophilizate is prepared by
providing larval drone brood;
homogenizing same using a mixer, the content of dry matter in the homogenate not exceeding 22.5%;
freezing the homogenate for 2-3 hours at minus 35-40° C.;
sublimating in vacuum at a residual pressure of 0.1-0.6 mm of mercury for 40-48 hours, and
bringing the temperature of the lyophilizate to 20-30° C. by the end of lyophilization, whereby a powder with high hygroscopicity and residual moisture content of 1-3.5% is produced.
